(19) United States
(12) Patent Application Publication
(10) Pub. No.: US 2019/0290621 A1
(43) Pub. Date: Sep. 26, 2019

(54) COMPOSITIONS AND METHODS FOR TREATING HIV-1 LATENCY

(71) Applicants: Alexander B. Barnes, University City, MO (US); George Kyei, St. Louis, MO (US); Brice Albert, Cincinnati, OH (US)

(72) Inventors: Alexander B. Barnes, University City, MO (US); George Kyei, St. Louis, MO (US); Brice Albert, Cincinnati, OH (US)

(21) Appl. No.: 16/348,743

(22) PCT Filed: Nov. 9, 2017
(86) PCT No.: PCT/US17/60791

§ 371 Date: May 9, 2019

(51) Int. Cl. A61K 31/439; A61K 31/365; A61K 45/06; A61P 31/18; A61K 31/122

(52) U.S. Cl. CPC A61K 31/439; A61K 31/365; A61K 31/122; A61P 31/18; A61K 45/06

(57) ABSTRACT

Pharmaceutical compositions comprising a combination of a Class I isoform-selective histone deacetylase inhibitor and a protein kinase C modulator are described as well as methods of using these compositions for treating HIV-1 latency.

### GOVERNMENT LICENSE RIGHTS

This invention was made with Government support under grant DP2-GM119131 awarded by the National Institutes of Health. The Government has certain rights in the invention.

### FIELD OF THE INVENTION

The present invention generally relates to compositions comprising a combination of a Class I isoform-selective histone deacetylase inhibitor and a protein kinase C modulator as well as the use of these compositions for treating HIV-1 latency.

### BACKGROUND OF THE INVENTION

HIV-1 latency refers to an extremely stable but rare proviral reservoir formed within the resting memory of CD4+ T-cells. This reservoir is usually established within days of the initial infection. Additionally, this reservoir is capable of producing infectious virus when the host cell is reactivated upon exposure to an antigen. Upon activation, T-cells can undergo proliferation to generate effector cells that are capable of clearing the associated pathogen from the host. Activated T-cells usually die within a few weeks; however, some cells revert back to a resting state and persist as memory T-cells that are capable of being reactivated upon exposure to the same antigen. These cells are the primary reservoir the HIV latent provirus.

Viral latency in resting CD4+ T-cells remains the most important obstacle to HIV cure in patients on effective anti-retroviral therapy (ART) (3-5). This therapy reduces plasma viral load to undetectable levels (<20 copies per ml), resulting in immune recovery, increased lifespan and improved quality of life. Withdrawal of ART results in robust viral rebound from the latent T-cell reservoir even in patients who have remained undetectable for many years (6).

One strategy for eradicating latently infected T-cells is to stimulate replication of latent virus using small molecule latency reversing agents (LRAs) (7, 8). The idea behind this approach is that, once replication starts in the latent cells, viral cytopathic effects and antiviral immune responses will eliminate the infected cells (9). However, attempts to reverse latency and eliminate the reservoir with anti-T-cell receptor antibodies proved too toxic due to global T-cell activation (10, 11). Hence, efforts are now focused on LRAs that will stimulate viral replication without activating T-cells (9, 12). Two of the leading classes of small molecules that have been tested include histone deacetylase (HDAC) inhibitors and protein kinase C (PKC) modulators. Several HDAC inhibitors have been shown to reactivate HIV without global T-cell activation (13). Of these, valproic acid (VPA), romidepsin and suberoylanilide hydroxamic acid (SAHA, vorinostat) have reached the most advanced pre-clinical tests (14-18). Many PKC modulators have also been studied as candidates for LRA potential including ingenols (43), prostratin (44-46), 1,2 diacylglycerol analogs (47), and bryostatin-1 (19, 48, 49). Bryostatin-1, originally isolated from a marine bryozoan Bugula neritina, has been used in many phase I and phase II clinical trials as a therapeutic for many indications, including Alzheimer's disease, and now shows great LRA potential as a compound with the ability to increase HIV-1 mRNA levels close to those induced by T-cell activators in ex-vivo studies (41).

A critical shortcoming of most of the leading HDAC inhibitors, such as vorinostat, is their lack of isoform specificity (21), and thus, are highly prone to untoward side effects. HDACs are divided into Classes I, II, III and IV. The Class I HDACs includes HDAC1, -2, -3 and -8 while the Class II HDACs are HDAC4, -5, -6, -7, -9 and -10. Class III HDACs are sirtuins with a different mechanism that have not been associated with HIV-1 latency. The only member of Class IV is HDAC11 (22). There is accumulating evidence that HIV-1 latency requires Class I HDAC isoforms, especially HDAC1, -2 and -3, with HDAC3 being the most important (23, 24). Isoform-specific compounds targeting individual Class I HDAC isoforms are more desirable in eliminating untoward side effects (25-27). However, it is unclear whether isoform-specific compounds will be as effective in reactivating HIV from latency as their pan-HDAC counterparts.

Although neither HDAC inhibitors nor PKC modulators have been successful in reducing the viral reservoir to date, recent combination therapy of these two classes of LRAs has been shown to be more effective (19, 20) and the HDAC inhibitors have been integrated as an important tool in our arsenal to eliminate the latent reservoir. Nevertheless, there remains a need for compositions and treatment methods that are capable of stimulating viral replication without activating T-cells.

### SUMMARY OF THE INVENTION

Aspects of the present invention are directed to pharmaceutical compositions comprising a therapeutically effective amount of a histone deactylase (HDAC) inhibitor that is selective to Class I HDACs and a protein kinase C (PKC) modulator. Further aspects of the invention include a kit comprising a first pharmaceutical composition comprising a therapeutically effective amount of a HDAC inhibitor that is selective to Class I HDACs and a second pharmaceutical composition comprising a therapeutically effective amount of a PKC modulator.

Other aspects of the present invention are directed to methods for treating HIV-1 latency in a subject in need thereof. Various methods comprise administering to the subject a pharmaceutical composition comprising a therapeutically effective amount of the HDAC inhibitor that is selective to Class I HDACs and a PKC modulator. Other methods comprise administering to the subject a first pharmaceutical composition comprising a therapeutically effective amount of a HDAC inhibitor that is selective to Class I HDACs and a second pharmaceutical composition comprising a therapeutically effective amount of a PKC modulator.

Other objects and features will be in part apparent and in part pointed out hereinafter.

### BRIEF DESCRIPTION OF THE DRAWINGS

FIG. 1A: Results of a screening experiment that show largazole (SDL148) and largazole analogs reactivate HIV from latency in cells.

FIG. 1B: Results of a screening experiment that show largazole (SDL148) and largazole analogs reactivate HIV from latency in cells.

FIG. 1C: Graph of effective concentration (EC₅₀) of largazole (SDL148) tested to reactivate HIV from latency in cells.

FIG. 1D: Graph of effective concentration (EC₅₀) of a largazole analog (JMF1080) tested to reactivate HIV from latency in cells.

FIG. 1E: Graph of effective concentration (EC₅₀) of a largazole analog (SDL256) tested to reactivate HIV from latency in cells.

FIG. 1F: Results of a screening experiment that show largazole (SDL148) and largazole analogs reactivate HIV from latency in cells.

FIG. 1G: Results of an experiment that show largazole induces HIG Gag proteins.

FIG. 1H: Results of an experiment that show the toxicity profile of largazole and largazole analogs.

FIG. 2A: Results of an experiment that show largazole and largazole analogs inhibit Class I histone deacetylases in Jurkat cells.

FIG. 2B: Results of an experiment that show largazole and largazole analogs inhibit Class I histone deacetylases in primary T-cells.

FIG. 2C: Results of an experiment that show largazole and largazole analogs inhibit Class I histone deacetylases in HeLa cells.

FIG. 2D: Results of an experiment that show largazole and largazole analogs inhibit Class I histone deacetylases in HeLa cells.

FIG. 2E: Results of an experiment that show largazole and largazole analogs inhibit Class I histone deacetylases in HeLa cells.

FIG. 2F: Results of an experiment that show largazole and largazole analogs inhibit Class I histone deacetylases in primary T-cells.

FIG. 2G: Results of a chromatin immunoprecipitation (ChIP) assay.

FIG. 3A: Results of an experiment that show that a combination of largazole and protein kinase C modulators reactivates HIV.

FIG. 3B: Results of an experiment that show that a combination of largazole and protein kinase C modulators reactivates HIV.

FIG. 3C: Results of an experiment that show that a combination of largazole and protein kinase C modulators reactivates HIV.

FIG. 3D: Results of an experiment that show that a combination of largazole and protein kinase C modulators reactivates HIV.

FIG. 4A: Results of an experiment that show that a combination of largazole and protein kinase C modulators reactivates resting CD4+ T-cells.

FIG. 4B: Results of an experiment that show that a combination of largazole and protein kinase C modulators reactivates resting CD25 T-cells.

FIG. 4C: Results of an experiment that show that a combination of largazole and protein kinase C modulators reactivates resting CD69 T-cells.

Corresponding reference characters indicate corresponding parts throughout the drawings.

### DESCRIPTION OF THE PREFERRED EMBODIMENTS

In general, the present invention is directed to pharmaceutical compositions and methods for treating HIV-1 latency in a subject in need thereof. The pharmaceutical compositions generally comprise a histone deactylase (HDAC) inhibitor that is selective to Class I HDACs in combination with a protein kinase C (PKC) modulator. The methods comprise administering to the subject a therapeutically effective amount of the HDAC inhibitor that is selective to Class I HDACs and a therapeutically effective amount of the PKC modulator.

It has been unexpectedly discovered that the combination of at least one Class I isoform-selective HDAC inhibitor (such as largazole and analogs thereof as described further herein) and at least one PKC modulator (such as bryostatin-1, prostratin, and analogs thereof as described further herein) provides for a highly effective HIV-1 latency treatment strategy that leads to cell death through viral cytopathic effects and immune recognition following the reactivation of viral replication. It has been found that this combination of active ingredients induces unmatched HIV-1 reactivation levels while avoiding global T-cell activation in resting CD4+ T-cells.

Typically, the weight ratio of the HDAC inhibitor to PKC modulator in the pharmaceutical composition is at least about 1:1, at least about 2:1, at least about 3:1, at least about 5:1, at least about 10:1, at least about 20:1, at least about 50:1, or at least about 100:1. For example, the weight ratio of HDAC inhibitor to PKC modulator can range from about 1:1 to about 200:1, from about 1:1 to about 100:1, from about 1:1 to about 50:1, from about 1:1 to about 20:1, from about 1:1 to about 10:1, from about 1:1 to about 5:1, from about 1:1 to about 3:1, from about 2:1 to about 200:1, from about 2:1 to about 100:1, from about 2:1 to about 50:1, from about 2:1 to about 20:1, from about 2:1 to about 10:1, from about 2:1 to about 5:1, from about 2:1 to about 3:1, from about 3:1 to about 200:1, from about 3:1 to about 100:1, from about 3:1 to about 50:1, from about 3:1 to about 20:1, from about 3:1 to about 10:1, from about 3:1 to about 5:1, from about 5:1 to about 200:1, from about 5:1 to about 100:1, from about 5:1 to about 50:1, from about 5:1 to about 20:1, or from about 5:1 to about 10:1.

### Class I Isoform-Selective HDAC Inhibitors

In the search to find an effective HDAC inhibitor, isoform specificity is a highly desirable quality. Finding isoform-specific compounds may not only lead to fewer side effects, but will help define more clearly the molecular mechanisms whereby HDAC activity regulates HIV-1 latency. Typically, the HDAC inhibitor used in the compositions and methods of the present invention exhibit a selectivity to Class I HDACs that is at least about 20%, at least about 30%, at least about 40%, at least about 50%, at least about 60%, at least about 70%, at least about 80%, at least about 90%, or at least about 95%. For example, the selectivity to Class I HDACs can be in the range of from about 20% to about 95%, from about 30% to about 95%, from about 40% to about 95%, from about 50% to about 95%, from about 60% to about 95%, from about 70% to about 95%, from about 80% to about 95%, from about 20% to about 75%, from about 30% to about 75%, from about 40% to about 75%, from about 50% to about 75%, or from about 60% to about 75%.

One particular group of HDAC inhibitors that is highly selective to Class I HDACs include largazole and analogs of largazole. These compounds have been found to modify the HIV LTR chromatin landscape. As shown below, largazole is a 16-membered ring macrocyclic depsipeptide.

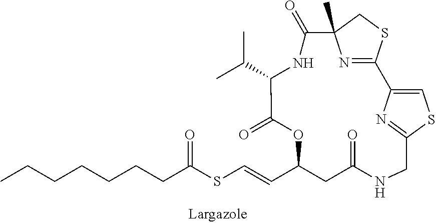 [SMILES: CCCCCCCC(=O)S/C=C/[C@@H]1CC(=O)NCc2nc(cs2)C2=N[C@@](C)(CS2)C(=O)N[C@@H](C(C)C)C(=O)O1]

The compound was initially isolated from the marine cyanobacterium Symploca sp. This natural product is a potent and Class I-selective HDAC inhibitor, with substantial potency against HDAC1, HDAC2, and HDAC3 in the picomolar range. Largazole and analogs thereof have been shown to differentially affect cancer cell growth, including colon and breast cancer cells and they have excellent bioavailability in mice (31).

Accordingly, in various embodiments, HDAC inhibitors useful for the compositions and methods of the present invention include largazole and analogs thereof. Largazole and various largazole analogs include compounds of general Formula I or pharmaceutically acceptable salts, solvates, prodrugs, or stereoisomers of these compounds:

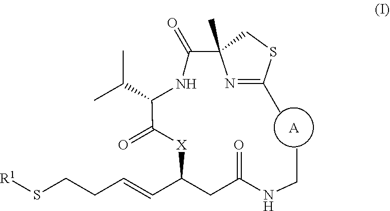

wherein X is 0 or NH;
R¹ is hydrogen or —C(O)R²;
R² is C₁-C₁₀ alkyl (branched or unbranched); and
A is 5- or 6-membered heterocyclic ring.

In various embodiments, R² is methyl, ethyl, propyl, butyl, pentyl, hexyl, or heptyl. In some embodiments, A is a 5- or 6-membered nitrogen-containing heterocyclic ring such as pyridine. In other embodiments, A is a 5- or 6-membered sulfur-containing heterocyclic ring such as thiazole.

In certain embodiments, the HDAC inhibitor compound of Formula I is selected from the group consisting of:

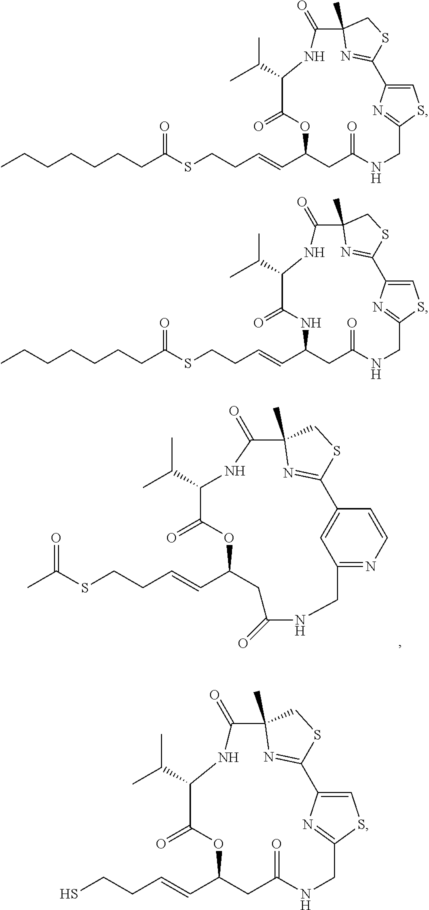

pharmaceutically acceptable salts, solvates, prodrugs, stereoisomers, and mixtures thereof.

Other largazole analogs are described in U.S. Pat. No. 8,217,076, the contents of which are hereby incorporated by reference. Accordingly, in various embodiments, the largazole analog comprises a compound of Formula XIIIa or a disulfide dimer of Formula XIIIb:

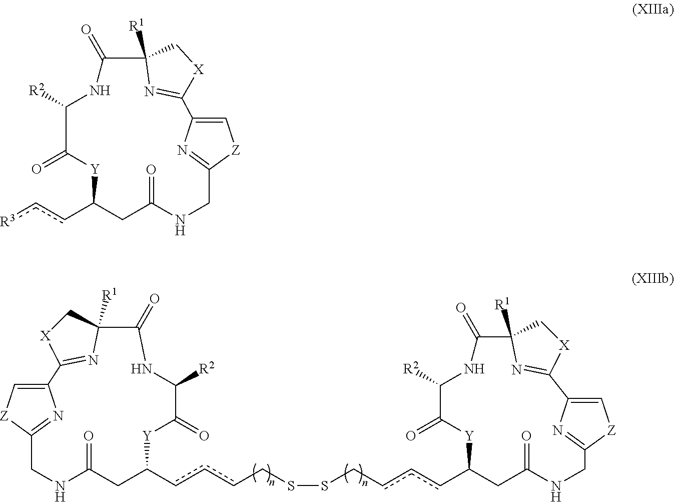

wherein in Formulas XIIIa and XIIIb:
X and Z are each independently S or O;
Y is NR or O;
R is H, lower alkyl, or lower arylalkyl;
R¹ is H, lower alkyl or lower arylalkyl;
R² is lower alkyl, isopropyl, n-propyl, cyclopropyl, isobutyl, n-butyl, sec-butyl, or tert-butyl;
R³ is H, (CH₂)ₙCO₂H, (CH₂)ₙCONHR, (CH₂)ₙCONHOH, (CH₂)ₙSR⁴, SR⁵, (CH₂)ₙNHC(O)CH₂SR or

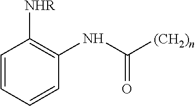

R⁴ is H, acyl, octanoyl, a higher acyl derivative, or SR;
R⁵ is lower alkyl or lower aryl; and
n is at least 1 (e.g., 1, 2, 3, 4 or 5); or a pharmaceutically acceptable salt, solvate, prodrug, or stereoisomer thereof.

In certain embodiments, the largazole analog comprises a compound of Formula XIIIa that is selected from the group consisting of:

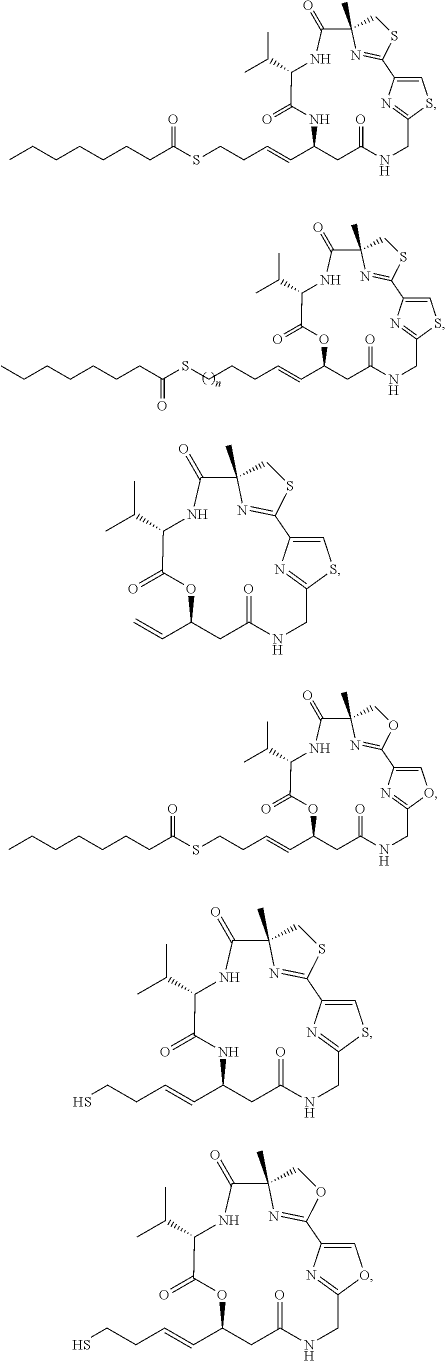

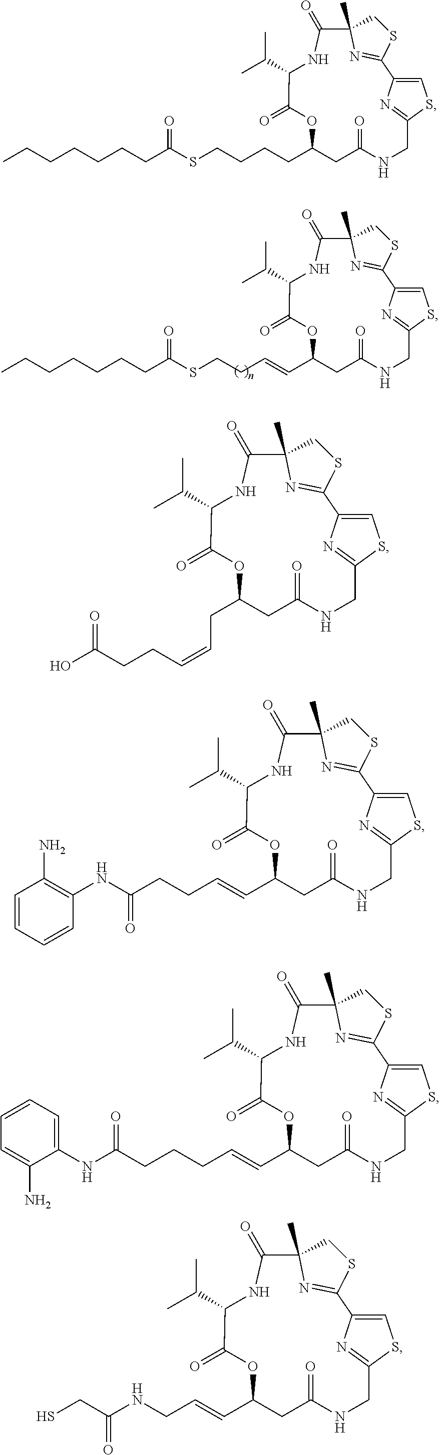

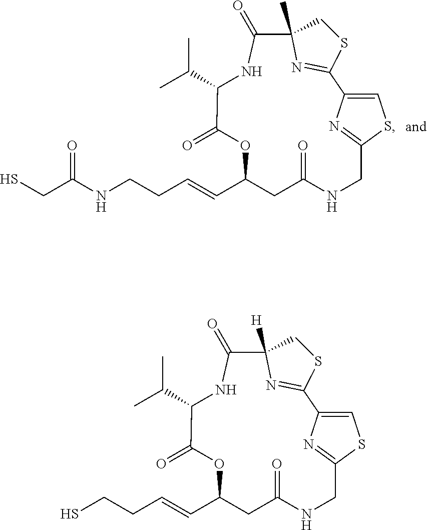

wherein n is at least 1 (e.g., 1, 2, 3, or 4).

In various embodiments, the largazole analog comprises a compound of Formula XIVa or a disulfide dimer of Formula XIVb, wherein the substituents are defined as above for compounds of Formulas XIIIa and XIIIb.

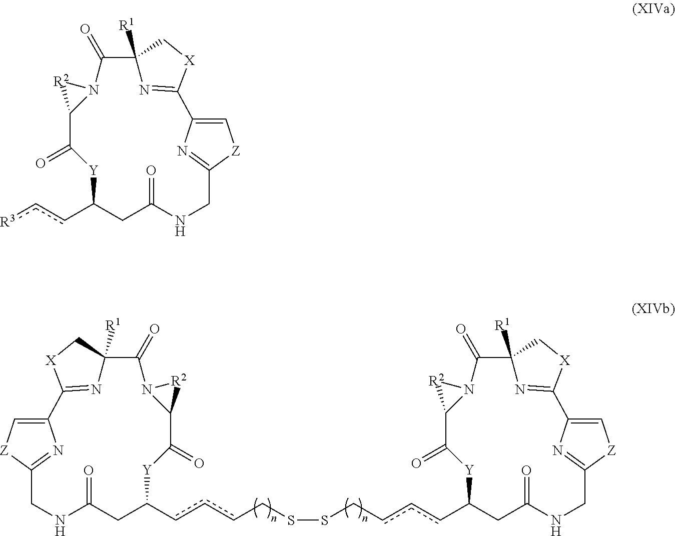

An exemplary compound of Formula XIVa has the structure shown below.

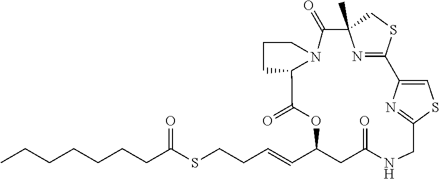 [SMILES: CCCCCCCC(=O)SCC/C=C/[C@@H]1CC(=O)NCc2nc(cs2)C2=N[C@@](C)(CS2)C(=O)N2CCC[C@H]2C(=O)O1]

In certain embodiments, the largazole analog comprises a compound of Formula XVa or a disulfide dimer of Formula XVb wherein the substituents are defined as above for compounds of Formula XIIIa and XIIIb.

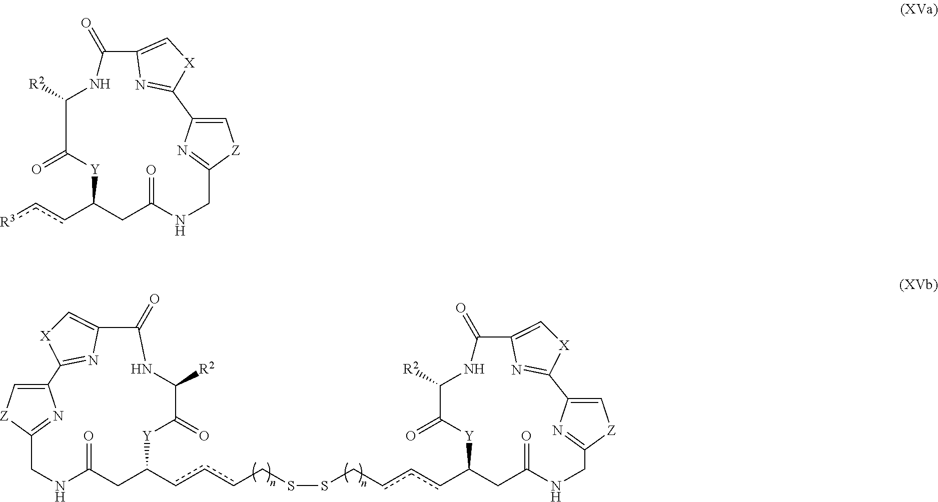

An exemplary compound of Formula XVa has the structure shown below.

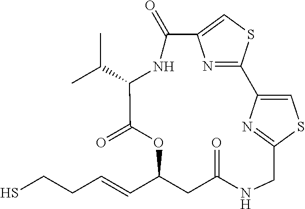 [SMILES: CC(C)[C@@H]1NC(=O)c2csc(n2)-c2csc(n2)CNC(=O)C[C@@H](/C=C/CCS)OC1=O]

In various embodiments, the largazole analog comprises a compound of Formula XVIa or a disulfide dimer of Formula XVIb:

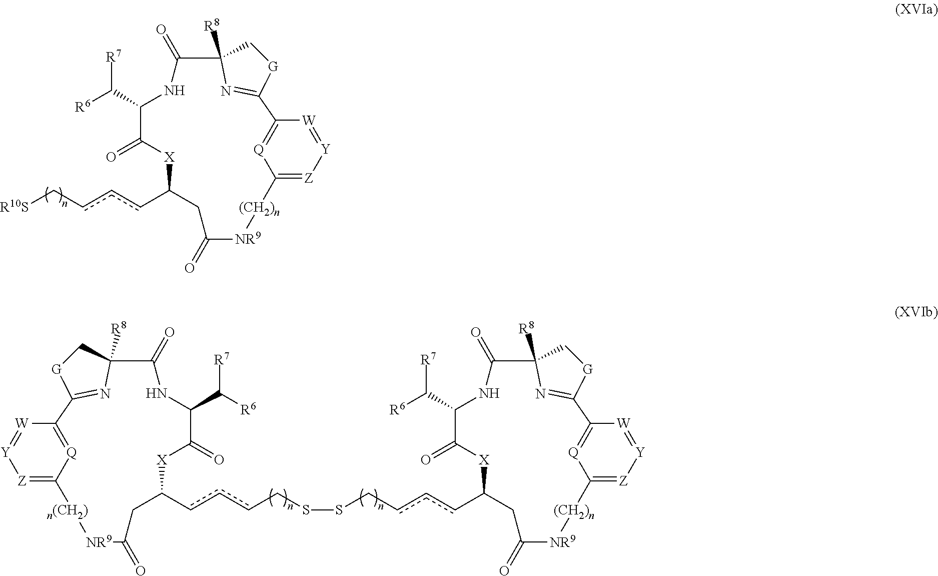

wherein in Formulas XVIb and XVIa:
X is O or NR¹²;
G is S, O, or NR¹²;
Q, W, Y, and Z are independently, N or CH, wherein at least one of Q, Y, Y, and Z is CH;
R⁶ is and R⁷ are each independently H or lower alkyl;
R⁸ is H, lower alkyl, or lower arylalkyl;
R⁹ is H or lower alkyl;
R¹⁰ is octanoyl, C(O)R¹¹;
R¹¹ is lower alkyl, lower aryl, or lower arylalkyl;
R¹² is H, lower alkyl, or lower arylalkyl; and
n is 0, 1, 2, or 3
or a pharmaceutically acceptable salt, solvate, prodrug, or stereoisomer thereof.

Exemplary compounds of Formula XVIa include those having the following structures:

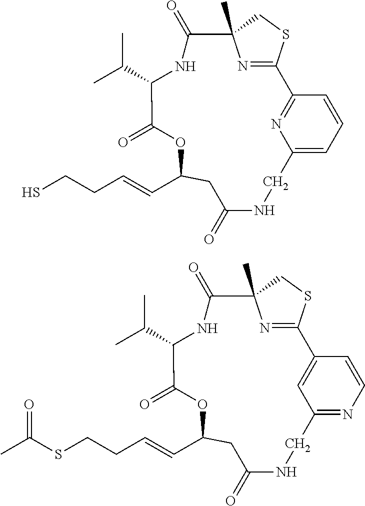

In the compounds of Formula XIII, XIV, XV, and XVI, the designation of one line parallel to a dotted line represents an optional double bond. That is, the bond can be a single bond or a double bond. When a double bond is present, the alkene may have either a cis- or trans-configuration.

Still other largazole analogs useful in the compositions and methods of the present invention include largazole mimetics such as those described in International Application No. PCT/US2016/030995, which is incorporated herein by reference. These largazole mimetics are compounds of general Formula XVII:

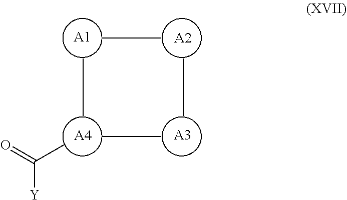

wherein A1 and A2 are each independently L-Pro, D-Pro, L-NMe-AA or D-NMe-AA; A3 is a natural or unnatural alpha-amino acid; and A4 is L- or D-aspartic wherein the α-carboxyl group is unprotected (Y═OH) or wherein the α-carboxyl group has been converted to an ester or amide derivative.

Preferably, the largazole mimetics include compounds of Formula XVIIa, XVIIb, XVIIc, and/or XVIId or a pharmaceutically acceptable salt, solvate, prodrug, or stereoisomer thereof, having a diproline subunit at Al-A2, a naturally occurring L-amino acid at A3, and L- or D-aspartic acid (or ester or amide derivative thereof) at A4 as shown below:

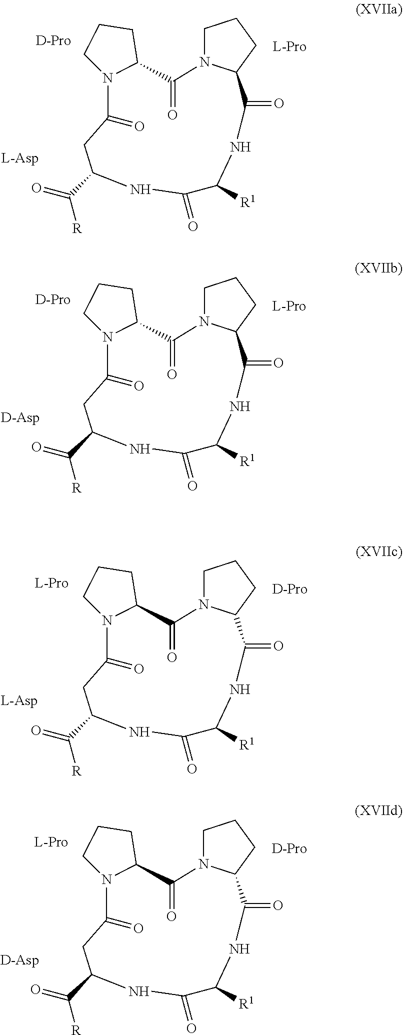

wherein in Formulas XVIIa, XVIIb, XVIIc, and XVIId:
R is hydroxyl, substituted or unsubstituted alkoxy, substituted or unsubstituted aryloxy, substituted or unsubstituted arylalkyloxy, or substituted or unsubstituted amino; and R¹ is a naturally occurring L-amino acid.

Compounds of Formulas XVIIa, XVIIb, XVIIc, and XVIId are cyclic tetrapeptides having a 13-membered ring derived from three alpha-amino acids and one beta-amino acid (i.e., α3β architecture). Compounds of Formula XVIIa and XVIIb have the dipeptide subunit D-Pro-L-Pro, whereas compounds of Formula XVIIc and XVIId have the dipeptide subunit L-Pro-D-Pro. The third amino acid is a naturally occurring L-amino acid, and the fourth amino acid is a β-amino acid which is L-Asp (compounds XVIIa and XVIIc) or D-Asp (compounds XVIIb and XVIId).

The R¹ group in these compounds derives from the third amino acid. In various embodiments, R¹ is H (Gly), Me (Ala), isopropyl (Val), isobutyl (Leu), or sec-butyl (Ile). Side chains from other natural amino acids are also included. In preferred embodiments, R¹ is isopropyl (Val).

The cyclic tetrapeptide also has a side chain that is a carboxyl group, corresponding to the α-carboxyl group of L- or D-aspartic acid, or a derivative thereof. The carboxyl group can be converted, for example, into an ester. Accordingly, in Formulas XVIIa, XVIIb, XVIIc, and XVIId, R can be hydroxy, substituted or unsubstituted alkoxy, substituted or unsubstituted aryloxy, or substituted or unsubstituted arylalkyloxy. In various embodiments, R is hydroxy, substituted or unsubstituted C₁-C₆ alkoxy, substituted or unsubstituted phenoxy, or substituted or unsubstituted benzyloxy. In certain embodiments, R is hydroxy or benzyloxy.

The carboxyl group can also be converted into an amide. Accordingly, in Formulas XVIIa, XVIIb, XVIIc, and XVIId, R can be amino (NH₂) or substituted amino. In various embodiments, R is substituted amino having the formula —NH—(CH)ₙ—R², where R² is OH, SR³, SOR³, SO₂R³, NR³, CO₂R³, C(O)NHOR³, S—S(CH₂)ₙNH₂, —NH(CH₂)ₙS—S(CH₂)ₙNHPO(OR⁴)₂; R³ is hydrogen or C₁-C₆ alkyl (e.g., methyl or ethyl); R⁴ is hydrogen or phenyl; and n is a number from 2 to 5 (e.g., n can be 2 to 3).

The above mentioned HDAC inhibitors can be prepared according to known techniques including those disclosed in the references mentioned herein.

### Protein Kinase C Modulators

As noted, the pharmaceutical compositions of the present invention comprise at least one PKC modulators in combination with the HDAC inhibitor as described herein. PKC modulators which have been found to particularly useful in the present invention include, for example, bryostatin-1, prostratin, and analogs of these compounds.

In various embodiments, the protein kinase C modulator comprises bryostatin-1 or an analog thereof. The structure of bryostratin-1 is shown below:

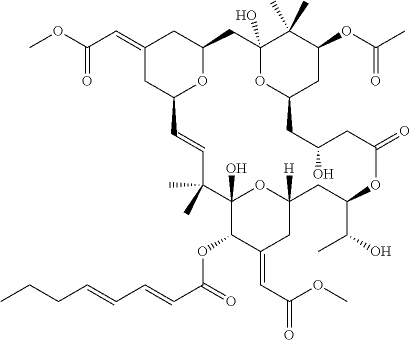

Various analogs of bryostratin-1 analog include compounds having the structure of Formula IIa or IIb, or a pharmaceutically acceptable salt, solvate, prodrug or stereoisomer thereof:

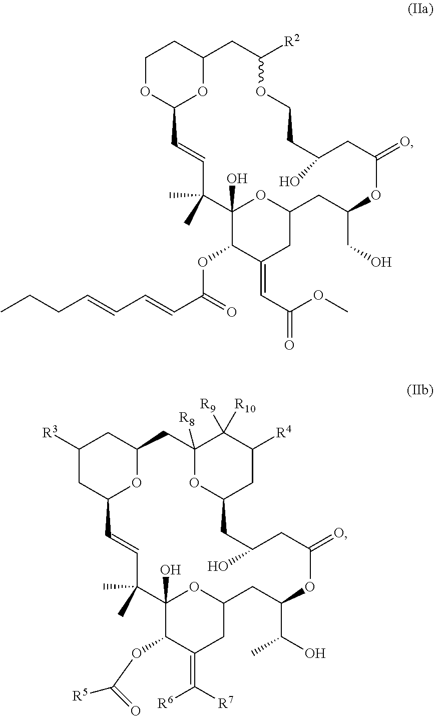

wherein
R² is hydrogen, methyl, ethyl, propyl, isopropyl, tert-butyl, phenyl, or —(CH₂)₃p-Br-Ph
R³ is hydrogen, ═CH₂, ═CHC(O)OCH₂, —CHCO₂CH₃, —CH₂CHCH₂, or —CH₂CO₂(CH₂)₂;
R⁴ is hydrogen, hydroxyl, —OC(O)CH₃, —OC(O)C(CH₃)₃, or —OC(O)C(CH₂)₂CH₃;
R⁵ is hydrogen, —CH₃, —(CH₂)₂CH₃, —(CH)₄(CH₂)₂CH₃, or —C₇H₁₅;
R⁶ and R⁷ are each independently hydrogen or —C(O)OCH₃;
R⁸ is hydrogen or hydroxyl; and
R⁹ and R¹⁰ are each independently hydrogen or methyl.

In certain embodiments, the bryostratin analogs are compounds of the following structures:

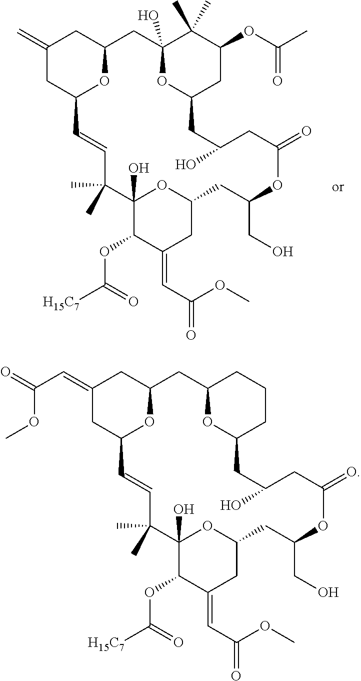

Other bryostratin analogs are described in Wender, P. A., Curr. Drug Disc. Tech., 2004, 1, 1-11; Wender, P. A., Proc. Natl. Acad. Sci. U.S.A. 1998, 95, 6624-6629; Wender, P. A., et al. JACS. 2002, 124, 13648-13649; U.S. Patent Publication Nos. 2003/0233000 and 2008/0207742; and U.S. Pat. Nos. 4,611,066; 6,624,189; 7,256,286; and 8,735,609, the contents of which are hereby incorporated by reference.

In various embodiments, the protein kinase C modulator comprises prostratin or an analog thereof. The structure prostratin is shown below:

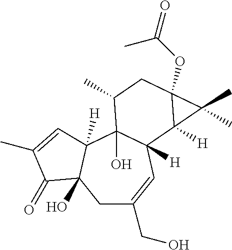

Various prostratin analog include compounds having the structure of Formula III or a pharmaceutically acceptable salt, solvate, prodrug or stereoisomer thereof:

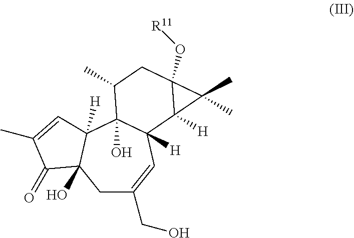

wherein
R¹¹ is C₁-C₆ alkyl (e.g., methyl, ethyl, propyl), —COCH₃, —COCH₂(Ph), or —(CH₂)₂(Ph).

Other prostratin analogs are described in Wender, P. A., et al. Science, 2008, 320, 649-652; Beans et al., Proc. Natl. Acad. Sci. U.S.A. 2013, 110, 11698-11703; and U.S. Pat. No. 8,816,122, the contents of which are hereby incorporated by reference.

The abovementioned PKC modulators can be prepared according to known techniques including those disclosed in the references mentioned herein.

As noted, other aspects of the invention are directed to a kit comprising a first pharmaceutical composition comprising a therapeutically effective amount of a HDAC inhibitor that is selective to Class I HDACs as described herein and a second pharmaceutical composition comprising a therapeutically effective amount of a PKC modulator also as described herein. In these embodiments, the HDAC inhibitor and PKC modulator are not necessary combined into a single pharmaceutical composition. Instead, the first and the second pharmaceutical composition but can be individual components of the kit. In various embodiments, the first and the second pharmaceutical compositions are the same type of dosage forms (e.g., oral dosage form such as tablets or capsules). In other embodiments, the first and the second pharmaceutical compositions are different types of dosage forms (e.g., combination of an oral and an intravenous dosage forms).

Pharmaceutical compositions containing one or more of the compounds described herein can be formulated in any conventional manner. Proper formulation is dependent in part upon the route of administration selected. Routes of administration include, but are not limited to, oral, parenteral (e.g., intravenous, intra-arterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration.

Pharmaceutically acceptable excipients for use in the compositions of the present invention are selected based upon a number of factors including the particular compound used, and its concentration, stability and intended bioavailability; the subject, its age, size and general condition; and the route of administration.

The pharmaceutical compositions can be formulated, for example, for oral administration. The pharmaceutical compositions can be formulated as tablets, dispersible powders, pills, capsules, gel-caps, granules, solutions, suspensions, emulsions, syrups, elixirs, troches, lozenges, or any other dosage form that can be administered orally. Pharmaceutical compositions can include one or more pharmaceutically acceptable excipients. Suitable excipients for solid dosage forms include sugars, starches, and other conventional substances including lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, microcrystalline cellulose, colloidal silicon dioxide, croscarmellose sodium, talc, magnesium stearate, and stearic acid. Further, such solid dosage forms can be uncoated or can be coated to delay disintegration and absorption.

The pharmaceutical compositions can also be formulated for parenteral administration, e.g., formulated for injection via intravenous, intra-arterial, subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, non-volatile silicones, adhesives, bulking agents, flavorings, sweeteners, adsorbents, binders, disintegrating agents, lubricants, coating agents, and antioxidants.

As noted, the present invention includes methods of treating HIV-1 latency. In various embodiments, the method comprises administering to the subject a pharmaceutical composition comprising a therapeutically effective amount of a HDAC inhibitor that is selective to Class I HDACs and a PKC modulator as described herein. In other embodiments, the method comprises administering to the subject a first pharmaceutical composition comprising a therapeutically effective amount of a HDAC inhibitor that is selective to Class I HDACs (as described herein) and a second pharmaceutical composition comprising a therapeutically effective amount of a PKC modulator (as described herein).

In various embodiments, the methods can further comprise administering antiviral agents, such as those currently used in highly active antiretroviral therapy (HAART).

As used herein, “lower alkyl” or “lower alkyl moieties” contain from 1-12 carbon atoms, “lower aryl” or “lower aryl moieties” contain from 6-12 carbon atoms, and “lower arylalkyl” or “lower arylalkyl moieties” contain from 7-12 carbon atoms. In a preferred embodiment, lower alkyl refers to a C₁₋₇ alkyl, lower aryl refers to a C₆₋₁₀ aryl, and lower arylalkyl refers to a C₇₋₁₁ aralkyl. Included are substituted derivatives of lower chain alkyl, aryl and arylalkyl moieties, wherein the substituent is selected from (but are not limited to) one or more of the following chemical moieties: —OH, —OR, —COOH, —COOR, —CONH₂, —NHR, —NRR, —SH, —SR, —SO₂R, —SO₂H, —SOR, —PO₃R, —OPO₃R, and halogen (including F, Cl, Br and I), wherein each occurrence of R is independently selected from a lower chain alkyl, aryl or arylalkyl moiety. Moreover, cyclic lower chain alkyl, aryl and arylalkyl moieties of the invention include naphthalene, as well as heterocyclic compounds such as thiophene, pyrrole, furan, imidazole, oxazole, thiazole, pyrazole, 3-pyrroline, pyrrolidine, pyridine, pyrimidine, purine, quinoline, isoquinoline and carbazole.

As used herein, the term “prodrug” refers to a derivative of a compound that can hydrolyze, oxidize, or otherwise react under biological conditions (in vitro or in vivo) to provide a compound as described herein. Prodrugs may only become active upon some reaction under biological conditions, but they may have activity in their unreacted forms. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs and their uses are well known in the art (see, e.g., Berge, et al. 1977 J. Pharm. Sci. 66:1-19). Prodrugs can typically be prepared using well-known methods, such as those described in Burger's Medicinal Chemistry and Drug Discovery (1995, Manfred E. Wolff ed., 5thed. 172-178, 931-932).

“Pharmaceutically acceptable salt” as used herein refers to salts of the compounds described herein which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

“Subject” as used herein refers to a mammal, including both human and non-human mammals. Preferred subjects are human subjects.

“Treat”, “treating”, and “treatment” refer to a method of alleviating or abating a disease and/or its attendant symptoms.

As used herein, the following abbreviations and definitions are: “HDAC” refers to histone deacetylase; “PKC” refers to protein kinase C; “SAHA” refers to suberoylanilide hydroxamic acid; “TNF-alpha” refers to tumor necrosis factor alpha;

Having described the invention in detail, it will be apparent that modifications and variations are possible without departing from the scope of the invention defined in the appended claims.

### EXAMPLES

The following non-limiting examples are provided to further illustrate the present invention.

### Example 1

### Materials and Methods

Tumor necrosis factor alpha (TNF-alpha), bryostatin, vorinostat (SAHA), phorbol 12-myristate 13-acetate (PMA), CD25 antibodies for fluorescent-activated cell sorting (FACS) and ionomycin were obtained from Sigma-Aldrich (St. Louis, Mo.). Anti-acetylated histone H3 antibody was obtained from Thermo Scientific (Waltham, Mass.).

JLAT 10.6 and JLAT 9.2 cells were obtained from the National Institutes of Health AIDS Reagent Program, Division of AIDS and National Institute of Allergy and Infectious Diseases. JLAT and Jurkat cells were maintained in Roswell Park Memorial Institute (RPMI) medium supplemented with fetal bovine serum (FBS), L-glutamine, sodium pyruvate, and penicillin or streptomycin.

CD4+ T-cells were obtained from HIV-1 infected patients.

### Example 2

### Primary Cell Isolation and Culture

Peripheral blood mononuclear cells were isolated using density gradient centrifugation through a Ficoll-Hypaque gradient (GE Healthcare). CD4+ T-cells were isolated using the EASYSEP CD4+ T-Cell enrichment kit (STEMCELL Technologies; Vancouver, Calif.). Resting CD4+ T lymphocytes were further enriched by depletion of cells expressing CD25 by negative selection (STEMCELL Technologies). The remaining CD4+ lymphocytes were verified by flow cytometry and were typically greater than 98%. Isolated cells were maintained in RPMI supplemented with 10% FBS.

### Example 3

### Screen of HDAC Inhibitors for Reversing HIV-1 Latency

A series of structurally diverse HDAC inhibitors were screened for their ability to reactivate HIV-1 from latency. The structures of the HDAC inhibitors screened are shown below.

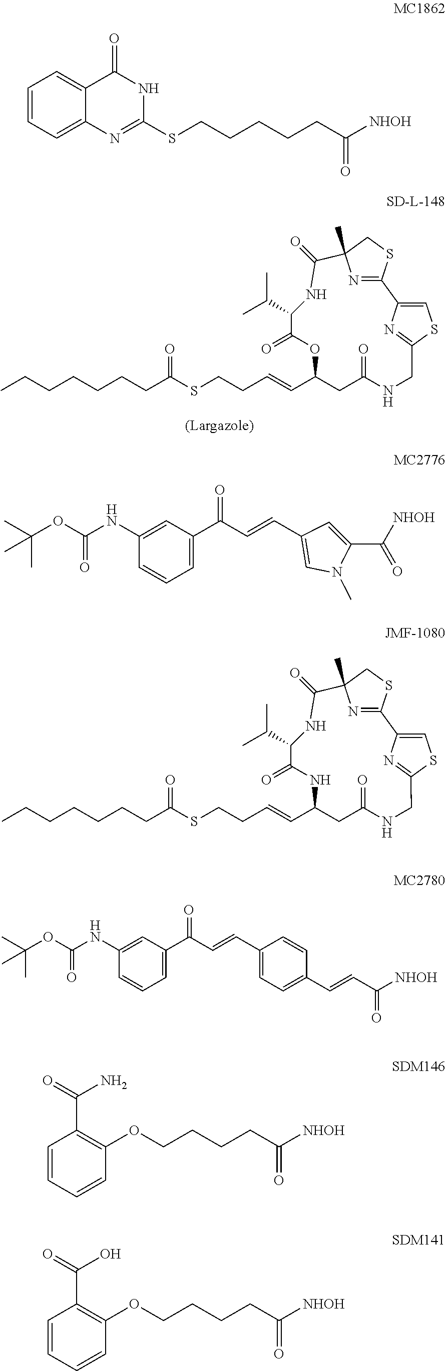

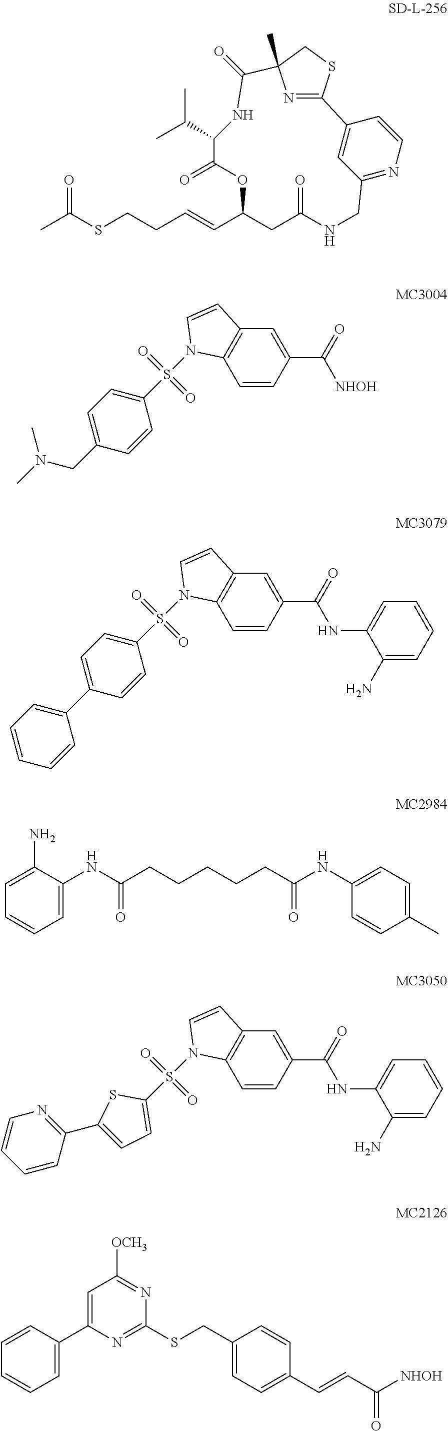

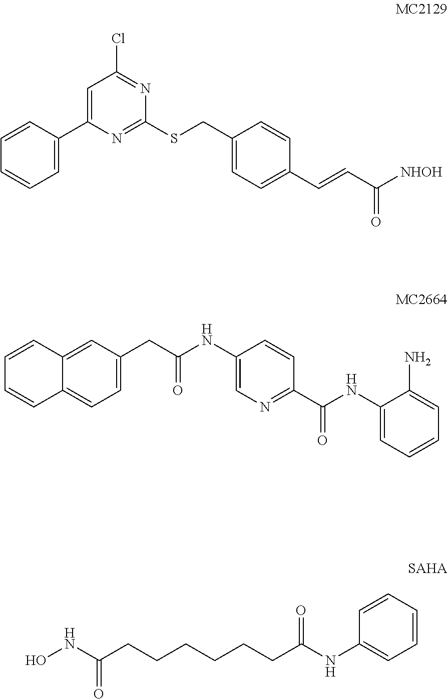

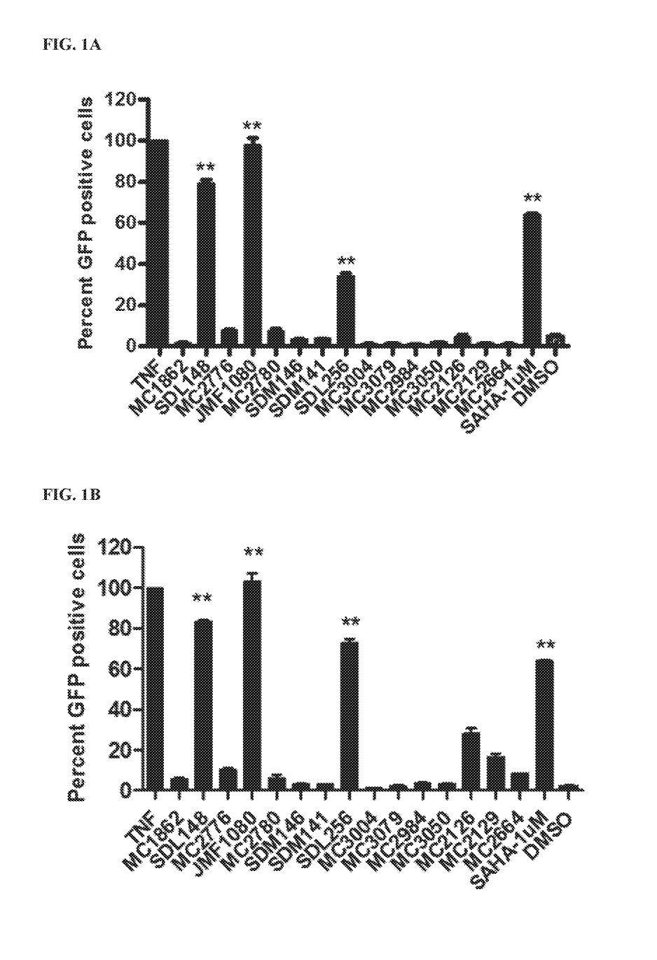

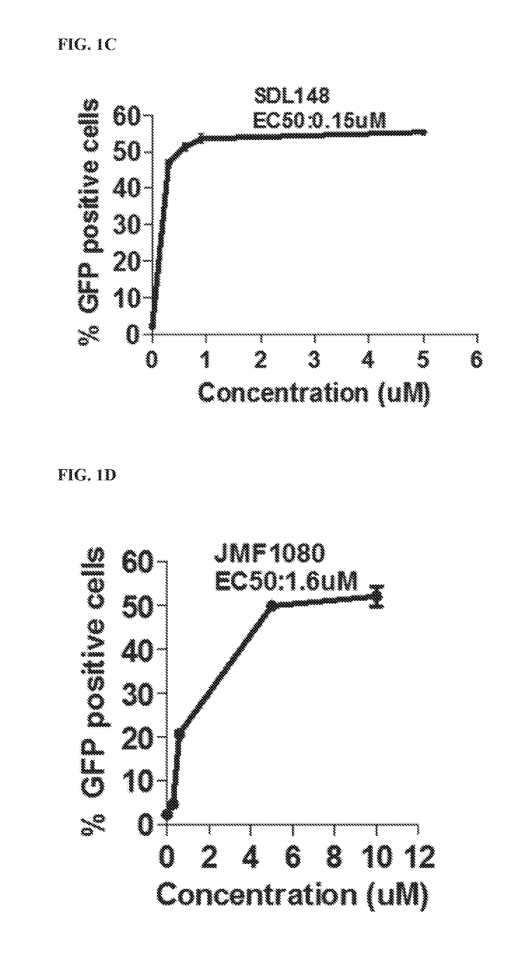

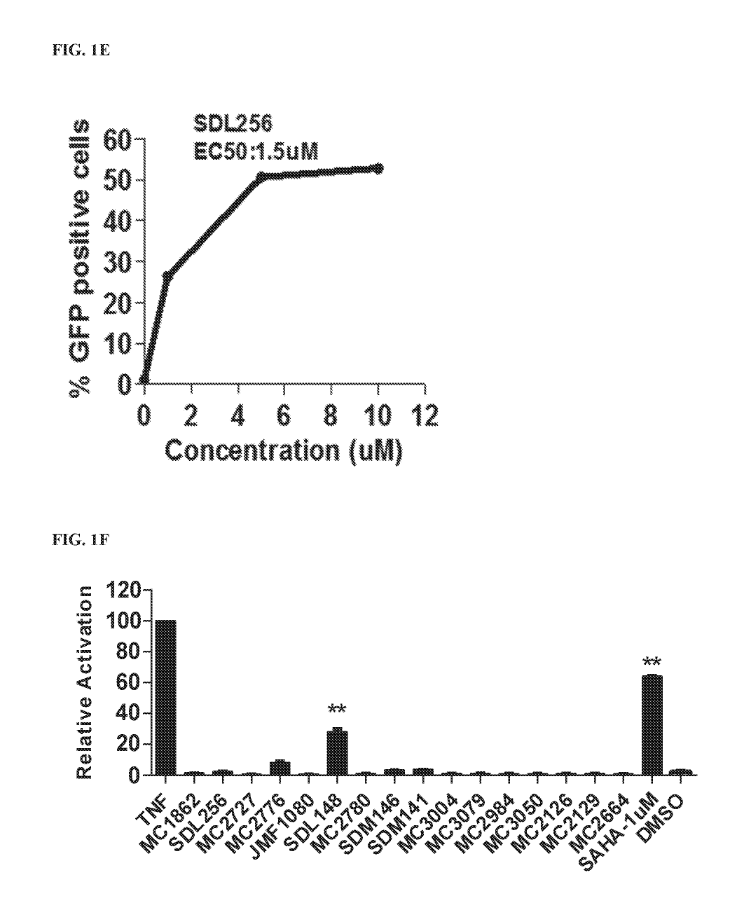

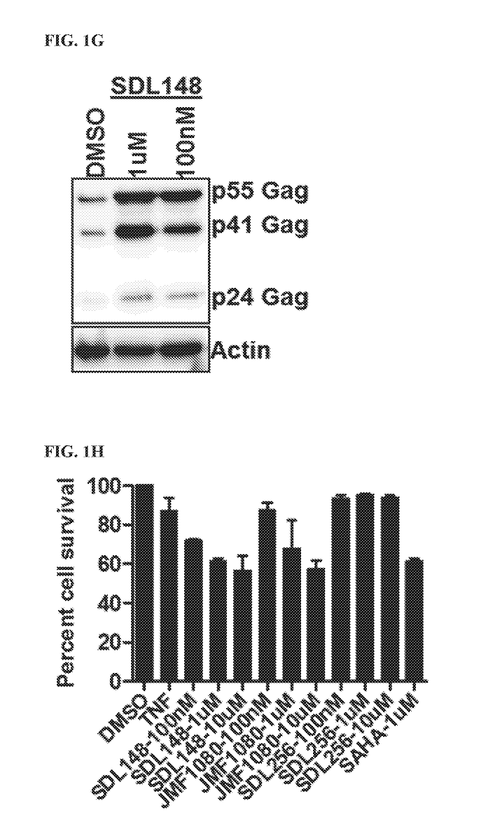

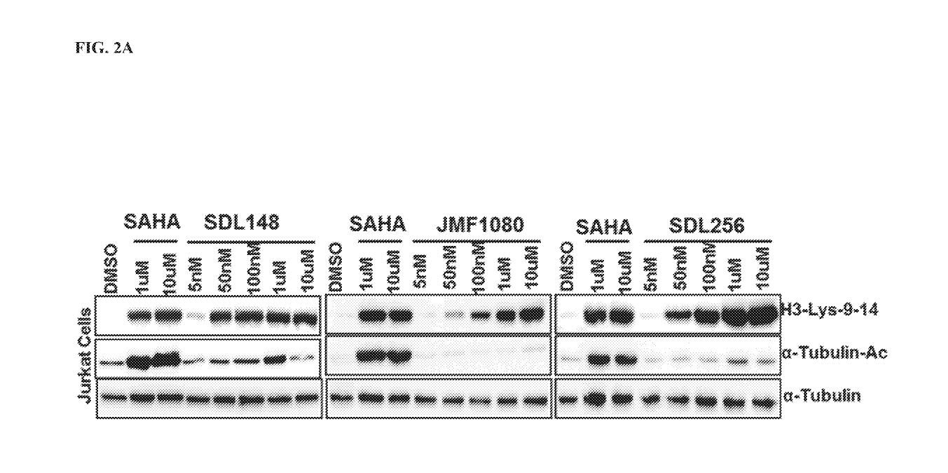

For screening purposes, JLAT10.6 cells, a T-cell line that has been widely used for HIV-1 reactivation studies (32-34) were utilized. In this cell line, HIV-1Δenv from the NL4-3 backbone with GFP replacing the Nef gene is stably integrated into the genome and is expressed at undetectable levels. Upon HIV reactivation with latency reversing agents (LRAs), viral production leads to expression of GFP that was measured by fluorescent activation cell sorting (FACS) analysis using a Becton Dickinson FACS machine. Analysis was performed using BD CELLQUEST PRO software (BD Biosciences; Franklin Lakes, N.J.). The stronger the HIV reactivation, the higher the percentage of GFP-positive cells observed. JLAT10.6 cells were incubated with HDAC inhibitors at concentrations of 1 and 10 μM for 24 hours, using TNF-α and SAHA (Vorinostat) as positive controls. The screening results are presented in FIG. 1A and FIG. 1B (data indicate means, and error bars indicate±SEM (n≥3), (ANOVA)).

Compounds SDL148 (largazole), JMF1080 and SDL256 (largazole analogs) were found to be comparable or more potent than vorinostat in reactivating HIV from latency.

Next, we determined the EC50 and toxicity levels for each of the three largazole compounds (see FIGS. 1C, 1D, and 1E). With an EC50 of 0.15 μM, SDL148 was found to be most potent among the three HDAC inhibitors followed by JMF1080 (1.5 μM) and SDL256 (1.6 μM). Consistent with its superior potency, SDL148 was the only compound able to reactivate HIV expression significantly in the JLAT10.6 cells when used at 100 nM (see FIG. 1F). Thus, we utilized SDL148 for most reactivation studies in subsequent experiments.

We then confirmed that the GFP expression seen with FACS corresponds to enhanced expression of HIV Gag as assessed by a Western blot after incubation of JLAT10.6 cells with SDL148 for 24 h (FIG. 1G).

To determine the toxicity of these compounds on the JLAT10.6 cells, we used a MTT (3-(4,5-dimethylthiazol-2-yl)-2,5-diphenyltetrazolium) cell-proliferation assay (35). Briefly, the MTT assay is a colorimetric assay that can be used to assay cell viability. The assay relies on the measurement of the presence of NAD(P)H-dependent cellular oxidoreductase enzymes, which are present in cellular mitochondria. The presence of these enzymes and thus the presence of active mitochondria is a reliable indicator of cellular viability. These enzymes reduce the MTT dye to formazan, which has a purple color, allowing the amount of these enzymes and by extension the amount of viable cells, to be quantified. After incubation with the indicated compounds for 3-4 hours, the MAT 10.6 cells were lysed using solublization buffer. The absorbance of each sample was reported by a microplate reader (TECAN Group Ltd. Mannedorf, SUI), using the Magellan software program at a wavelength of 570 nm with a reference wavelength of 750 nm.

We found the toxicity profile of the three compounds was similar to or better than that of SAHA (FIG. 1H). These results show that the largazoles can potently induce HIV-1 from latency with a toxicity profile comparable to that of the pan-HDAC inhibitor, SAHA.

### Example 4

### Isoform-Specific HDAC Inhibitors for Chromatin Remodeling at the HIV-1 Promoter

To determine the isoform specificity of largazole (SDL148) compared with largazole analogs JMF1080 and SDL256, we performed Western blots for acetylated histone H3 and acetylated tubulin in Jurkat cell lines and primary resting CD4+ T-cells. Jurkat cells were incubated with SDL148, JMF1080, and SDL256 at the indicated concentrations for 8 hours and Western blot performed for the levels of acetylated histone H3 as a measure of Class I HDAC inhibition, acetylated tubulin as a measure of Class II HDAC inhibition (31, 36), and tubulin. Resting CD4+ T-cells were incubated with vorinostat or the indicated largazole analog for 8 hours and Western blot performed.

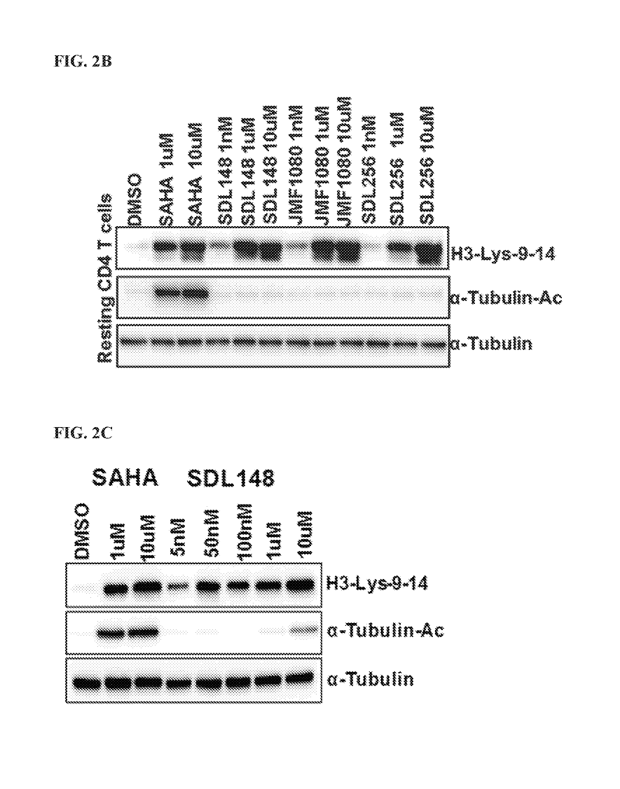

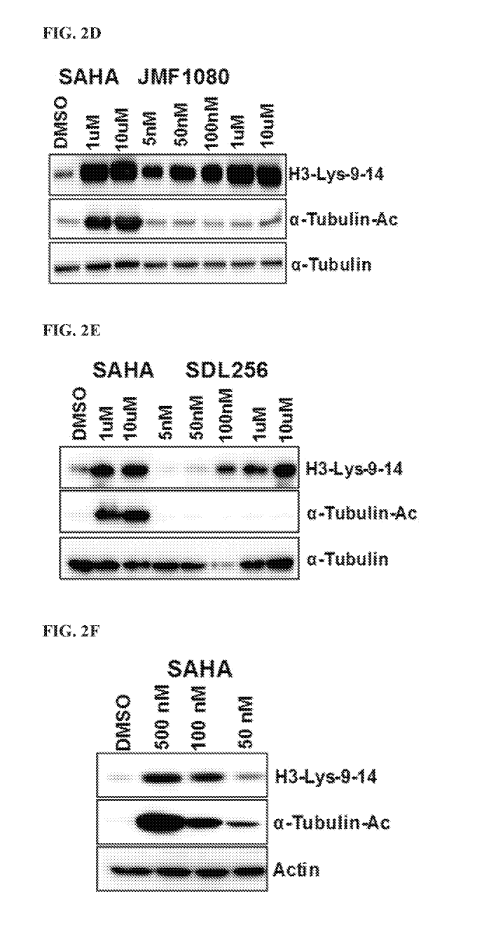

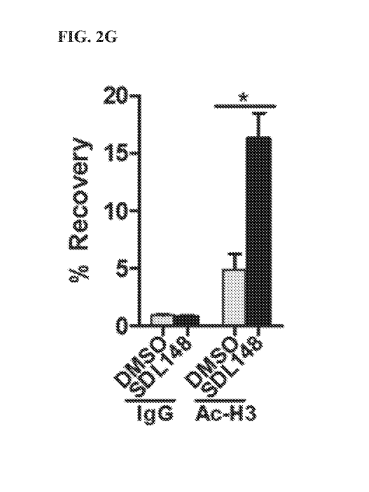

As shown in FIGS. 2A and 2B, the largazole compounds specifically enhanced histone H3 acetylation compared to SAHA, a pan-HDAC inhibitor that enhanced the acetylation of both histone H3 and tubulin. Consistent with the EC50 data in FIG. 1, SDL148 was the most potent, yielding levels of acetylated histone H3 at 50 nM similar to 1 μM of SAHA. The Class I HDAC inhibitory activity for the largazoles was not restricted to T-cells as they showed similar activity in HeLa cells (see FIGS. 2C, 2D, 2E, and 2F). While SAHA could induce the acetylation of tubulin at much lower concentrations, the largazoles did not induce detectable acetylated tubulin in primary T-cells with concentrations up to 10 μM. These data confirm that largazole and the largazole analogs are highly selective as Class I isoform HDAC inhibitors in T-cells.

Since histone H3 is acetylated by Class I HDACs and known to bind to the HIV LTR promoter, we determined H3 acetylation status of the HIV-1 LTR in JLAT10.6 cells. To do this, we added DMSO, or SDL148, to JLAT10.6 cells for 6 hours and performed chromatin immunoprecipitation (ChIP) assays. The ChIP assay was performed using a ChIP kit (ab500) (Abcam; Cambridge, Mass.). Briefly, an anti-rabbit IgG (Cell Signaling Technologies; Danvers, Mass.) was used as an isotype control. Actin was used as an internal control. Total chromatin (input) and the immunoprecipitated chromatin were used as templates and HIV-1 LTR was amplified by quantitative PCR using HIV-1 LTR (forward: 5-AGCCCTCAGATG CTACATATAAGCA-3, reverse: 5-TAG CCAGAGAGCTCCCAGGCTCAG A-3) and actin primers. Using ChIP, we recovered 15% of the DNA from SDL148-treated cells using anti-acetylated histone H3 antibody compared to less than 5% recovery with DMSO-treated cells, and less than 1% from the IgG-negative controls. See FIG. 2G (data indicate means, and error bars indicate±SEM (n≥3). *p<0.05, (student t test)). This shows that largazole actively remodel the chromatin at the HIV-1 LTR which could explain the reactivation of the virus. An alternative mechanism is modulation of lysines in Tat that impact its ability to interact with TAR of the HIV-1 LTR (37-39). Taken together, these findings confirm that largazole and the largazole analogs as HDAC inhibitors that preferentially target Class I HDACs with the ability to activate the HIV-1 LTR to overcome latency.

### Example 5

### Combination of Largazole and a PKC Modulator Shows HIV-1 Latency Activation

We used the JLAT 10.6 cell line to screen a series of PKC modulators to explore synergistic reactivation effects of HIV when combined with largazole (SDL148). The PKC modulators tested were bryostatin-1 and two bryostatin-1 analogs (SUW133 and SUW124) as shown below.

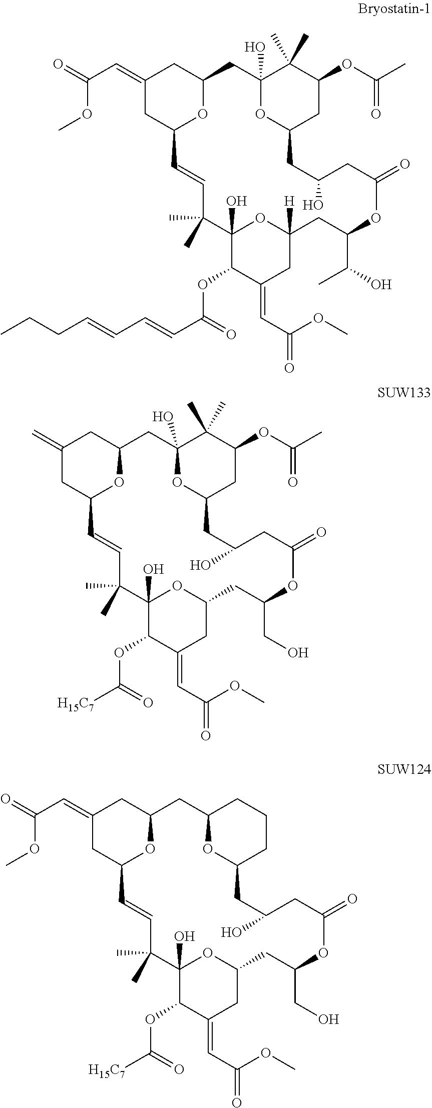

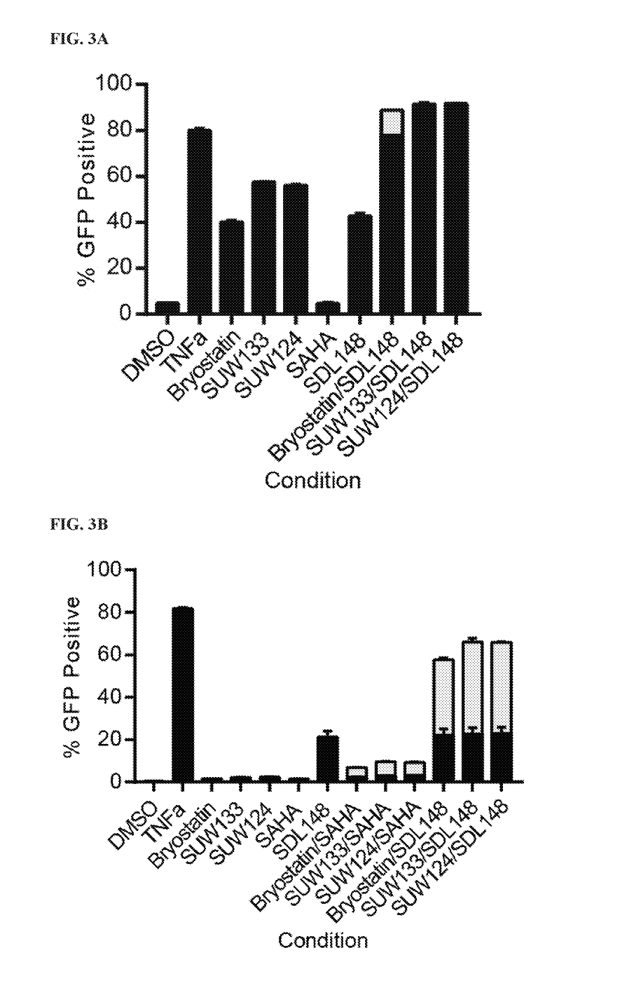

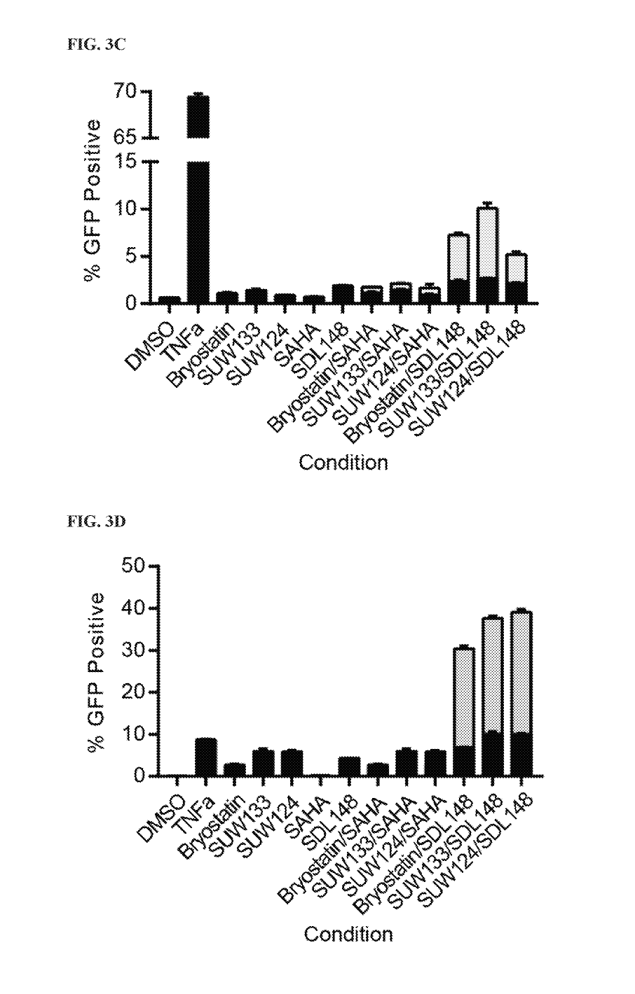

As in the screen of the HDAC inhibitors, FACS was used to assess HIV expression after activation with LRA combinations. FIGS. 3A, 3B, and 3C shows LRA activity in JLAT 10.6 cells at different PKC modulator and HDAC inhibitor concentrations. Any gray portion of the bar rising above the solid black bars in FIGS. 3A, 3B, 3C and 3D) represents a synergistic effect which is greater than expected from the addition of the percent of GFP positive cells produced from individual drug conditions. FIG. 3A shows activity with LRA concentrations of 10 nM PKC modulator and 100 nM HDAC inhibitor respectively. At 10 nM, individual conditions of the bryostatin and prostratin analogs were more effective in reactivating HIV-1 expression compared to bryostatin-1 as previously demonstrated (Beans 2012, and DeChristopher 2013). Combinations of the bryostatin and prostratin analogs with the targeted HDAC inhibitor, largazole, resulted in a percent of GFP positive cells which is greater than the positive control, TNF-α.

Combination of HDAC inhibitors with the PKC modulators concentration reduced to 1 nM still produced remarkable synergistic reactivation effects (FIG. 3B). The synergy of the PKC modulators with targeted largazole was much greater than with the untargeted vorinostat. Reducing the largazole concentration to 50 nM (FIG. 3C) continued to show impressive synergistic reactivation effects when combined with 1 nM concentrations of PKC modulator analogs.

To further assay the LRA effectiveness in initiation of HIV transcription at different integration zsites, we turned to the JLAT 9.2 cell line. JLAT 9.2 cells are more difficult to reactivate due to the location of the HIV-1 integration site within the genome (53). The synergistic reactivation experiments conducted with the JLAT 9.2 cell line included similar LRA combinations as those used in the JLAT 10.6 cell line (FIG. 3D). Unprecedented levels of reactivation were observed in the 9.2 cell line when the PKC modulator analogs were combined with largazole, at 10 nM and 100 nM respectively. Remarkably, these combinations induced a percent of GFP positive cells four times greater than even TNF-a, our positive control. Thus, this data shows that the combination of targeted class specific HDAC inhibitors, such as largazole, with bryostatin or prostratin analogs can be a highly potent HIV-1 LRA combination.

### Example 6

### Combination of HDAC Inhibitor and PKC Modulator Lead to HIV-1 Replication Without T-Cell Activation

CD4+ T-cells were isolated from an HIV(−) donor and activated with similar LRA conditions used in the JLAT experiments. The CD4+ T-cells were cultured for 24 hours with the following drug conditions in triplicate: 12.5 μL anti-CD3/CD28 beads, 50 ng/5 μg per mL of PMA/lonomycin, 1 nM bryostatin and prostratin analogs, and 100 nM largazole. Each trial contained one million CD4+ T-cells. After 24 hour activation, fluorescent labeled CD4, CD25, and CD66 antibodies were added and incubated at 4° C. for 20 minutes and then analyzed by FACS.

A total of 10,000 events were collected during FACS analysis. The events were gated based on FSC and SSC parameters of the true negative condition, i.e., cells cultured with DMSO and no antibody incubation, choosing the healthy cells. A second gate was then applied choosing a cut-off of activated versus non-activated cells based on the true negative control.

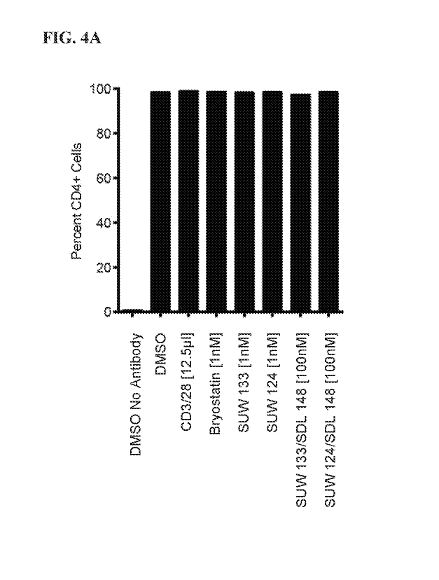

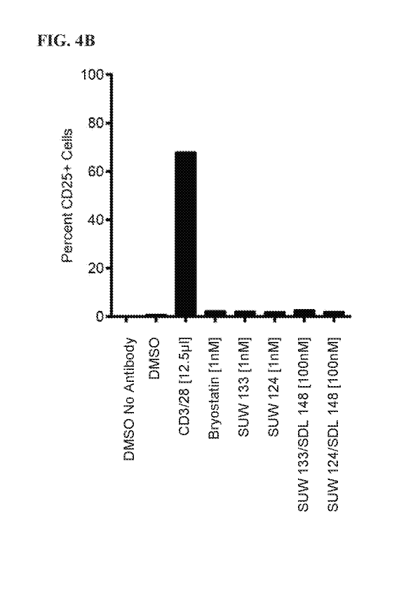

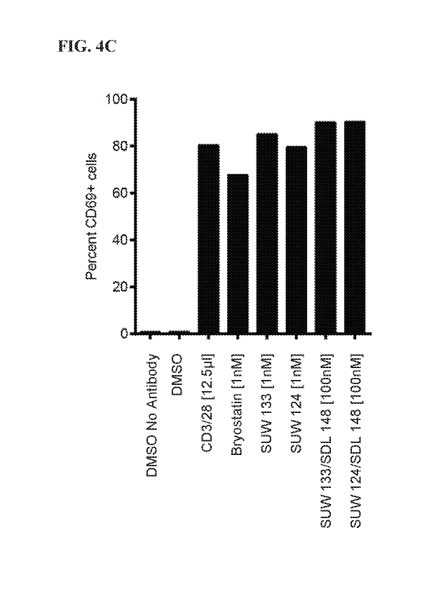

When CD4 positive cells are cultured with the concentrations indicated in FIGS. 4A, 4B, and 4C, the LRAs used in this study induce viral replication without causing global T-cell activation as indicated by the low levels of CD25+ T-cells. The results of percentage of cells that displayed CD4, CD25, and CD69 are shown in FIGS. 4A, 4B, and 4C, respectively.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and methods without departing from the scope of the invention, it is intended that all matter contained in the above description and shown in the accompanying drawings shall be interpreted as illustrative and not in a limiting sense.

### REFERENCES

1. Allfrey, V. G., et al., 1964. Acetylation and methylation of histones and their possible role in the regulation of RNA synthesis. Proc. Natl. Acad. Sci. U.S.A 51:786-794.

2. Choudhary, C., et al., 2009. Lysine acetylation targets protein complexes and co-regulates major cellular functions. Science 325:834-840.

3. Zhang, L., et al., 2000. Genetic characterization of rebounding HIV-1 after cessation of highly active antiretroviral therapy. J. Clin. Invest 106:839-845.

4. Chun, T. W., D et al., 2000. Relationship between pre-existing viral reservoirs and the re-emergence of plasma viremia after discontinuation of highly active anti-retroviral therapy. Nat. Med. 6:757-761.

5. Siliciano, J. M., and Siliciano, R. F. 2015. The Remarkable Stability of the Latent Reservoir for HIV-1 in Resting Memory CD4+ T Cells. J. Infect. Dis.

6. Davey, R. T., Jr., et al., 1999. HIV-1 and T cell dynamics after interruption of highly active antiretroviral therapy (HAART) in patients with a history of sustained viral suppression. Proc. Natl. Acad. Sci. U.S.A 96:15109-15114.

7. Archin, N. M., et al., 2014. Eradicating HIV-1 infection: seeking to clear a persistent pathogen. Nat. Rev. Microbiol. 12:750-764.

8. Richman, D. D., et al., 2009. The challenge of finding a cure for HIV infection. Science 323:1304-1307.

9. Xing, S., and Siliciano, R. F. 2013. Targeting HIV latency: pharmacologic strategies toward eradication. Drug Discov. Today 18:541-551.

10. van Praag, R. M., et al., 2001. OKT3 and IL-2 treatment for purging of the latent HIV-1 reservoir in vivo results in selective long-lasting CD4+ T cell depletion. J. Clin. Immunol. 21:218-226.

11. Kulkosky, J., et al., 2002. Intensification and stimulation therapy for human immunodeficiency virus type 1 reservoirs in infected persons receiving virally suppressive highly active antiretroviral therapy. J. Infect. Dis. 186:1403-1411.

12. Choudhary, S. K., and Margolis, D. M. 2011. Curing HIV: Pharmacologic approaches to target HIV-1 latency. Annu. Rev. Pharmacol. Toxicol. 51:397-418.

13. Shirakawa, K., Chavez, L., Hakre, S., Calvanese, V., and Verdin, E. 2013. Reactivation of latent HIV by histone deacetylase inhibitors. Trends Microbiol. 21:277-285.

14. Wei, D. G., et al., 2014. Histone deacetylase inhibitor romidepsin induces HIV expression in CD4 T cells from patients on suppressive antiretroviral therapy at concentrations achieved by clinical dosing. PLoS. Pathog. 10:e1004071.

15. Ylisastigui, L., et al., 2004. Coaxing HIV-1 from resting CD4 T cells: histone deacetylase inhibition allows latent viral expression. AIDS 18:1101-1108.

16. Archin, N. M., et al., 2009. Expression of latent human immunodeficiency type 1 is induced by novel and selective histone deacetylase inhibitors. AIDS 23:1799-1806.

17. Archin, N. M., et al., 2012. Administration of vorinostat disrupts HIV-1 latency in patients on antiretroviral therapy. Nature 487:482-485.

18. Archin, N. M., et al., 2014. HIV-1 expression within resting CD4+ T cells after multiple doses of vorinostat. J. Infect. Dis. 210:728-735.

19. Perez, M., et al., 2010. Bryostatin-1 synergizes with histone deacetylase inhibitors to reactivate HIV-1 from latency. Curr. HIV. Res. 8:418-429.

20. Laird, G. M., et al., 2015. Ex vivo analysis identifies effective HIV-1 latency-reversing drug combinations. J. Clin. Invest 125:1901-1912.

21. Butler, K. V., and Kozikowski, A. P. 2008. Chemical origins of isoform selectivity in histone deacetylase inhibitors. Curr. Pharm. Des 14:505-528.

22. Tripathy, M. K., et al., 2011. Epigenetic regulation of HIV-1 transcription. Epigenomics. 3:487-502.

23. Barton, K. M., et al., 2014. Selective HDAC inhibition for the disruption of latent HIV-1 infection. PLoS. One. 9:e102684.

24. Huber, K., et al., 2011. Inhibitors of histone deacetylases: correlation between isoform specificity and reactivation of HIV type 1 (HIV-1) from latently infected cells. J. Biol. Chem. 286:22211-22218.

25. Jones, R. B., et al., 2014. Histone deacetylase inhibitors impair the elimination of HIV-infected cells by cytotoxic T-lymphocytes. PLoS. Pathog. 10:e1004287.

26. Silvestri, L., et al., 2012. Histone deacetylase inhibitors: structure-based modeling and isoform-selectivity prediction. J. Chem. Inf. Model. 52:2215-2235.

27. Lucera, M. B., et al., 2014. The histone deacetylase inhibitor vorinostat (SAHA) increases the susceptibility of uninfected CD4+ T cells to HIV by increasing the kinetics and efficiency of postentry viral events. J. Virol. 88:10803-10812.

28. Ying, Y., et al., 2008. Synthesis and activity of largazole analogues with linker and macrocycle modification. Org. Lett. 10:4021-4024.

29. Hong, J., and Luesch, H. 2012. Largazole: from discovery to broad-spectrum therapy. Nat. Prod. Rep. 29:449-456.

30. Yu, M., et al., 2014. Largazole pharmacokinetics in rats by LC-MS/MS. Mar. Drugs 12:1623-1640.

31. Liu, Y., et al., 2010. Anticolon cancer activity of largazole, a marine-derived tunable histone deacetylase inhibitor. J. Pharmacol. Exp. Ther. 335:351-361.

32. Pion, M., et al., 2003. Transcriptional suppression of in vitro-integrated human immunodeficiency virus type 1 does not correlate with proviral DNA methylation. J. Virol. 77:4025-4032.

33. Spina, C. A., et al., 2013. An in-depth comparison of latent HIV-1 reactivation in multiple cell model systems and resting CD4+ T cells from aviremic patients. PLoS. Pathog. 9:e1003834.

34. Jordan, A., et al., 2003. HIV reproducibly establishes a latent infection after acute infection of T cells in vitro. EMBO J. 22:1868-1877.

35. Mosmann, T. 1983. Rapid colorimetric assay for cellular growth and survival: application to proliferation and cytotoxicity assays. J. Immunol. Methods 65:55-63.

36. Yuan, Z. 2009. Histone deacetylate activity assay, in Chromatin Protocols, S. P. Chellapan, Editor. Humana Press. p. 279-293

37. Ott, M., et al., 1999. Acetylation of the HIV-1 Tat protein by p300 is important for its transcriptional activity. Curr. Biol. 9:1489-1492.

38. Bres, V., et al., 2002. Tat acetyl-acceptor lysines are important for human immunodeficiency virus type-1 replication. J. Biol. Chem. 277:22215-22221.

39. Kumar, S., and Maiti, S. 2013. The effect of N-acetylation and N-methylation of lysine residue of Tat peptide on its interaction with HIV-1 TAR RNA. PLoS. One. 8:e77595.

40. Lassen, K. G., et al., 2012. A flexible model of HIV-1 latency permitting evaluation of many primary CD4 T-cell reservoirs. PLoS. One. 7:e30176.

41. Bullen, C. K., et al., 2014. New ex vivo approaches distinguish effective and ineffective single agents for reversing HIV-1 latency in vivo. Nat. Med. 20:425-429.

42. Varterasian M L, Mohammad R M, Shurafa M S, Hulburd K, Pemberton P A, Rodriquez D H, Eilender D S, Murgo A, Wall N, A.-K. A. Phase II trial of bryostatin 1 in patients with relapsed low grade non-Hodgkin's lymphoma and chronic lymphocytic leukemia. Clin. Cancer Res. 6, 825-828 (2000).

43. Warrilow, D., et al., 2006. HIV type 1 inhibition by protein kinase C modulatory compounds. AIDS Research and Human Retroviruses 22(9): 854-864.

44. Kulkosky, J., et al., 2001. Prostratin: activation of latent HIV-1 expression suggests a potential inductive adjuvant therapy for HAART. Blood, 98(10), 3006-3015.

45. Kulkosky, J., et al., 2004. Expression of latent HAART-persistent HIV type 1 induced by novel cellular activating agents. AIDS Research and Human Retroviruses, 20(5), 497-505.

46. Reuse, S., et al., 2009. Synergistic activation of HIV-1 expression by deacetylase inhibitors and prostratin: implications for treatment of latent infection. PLoS ONE, 4(6), e6093.

47. Hamer, D. H., et al., 2003. Rational design of drugs that induce human immunodeficiency virus replication. J. Virol., 77(19), 10227-10236.

48. del Real, G., et al., 2004. Statin inhibit HIV-1 infection by down-regulating rho activity. J. Exp. Med., 200(4), 541-547.

49. Mehla, R., et al., 2010. Bryostatin modulates latent HIV-1 infection via PKC and AMPK signaling but inhibits acute infection in a receptor independent manner. PLoS ONE, 5(6), e11160.

50. Beans, E. J., et al., 2013. Highly potent, synthetically accessible prostratin analogs induce latent HIV expression in vitro and ex vivo. PNAS, 110(29), 11698-11703.

51. DeChristopher, B. A., et al., 2012. Designed, synthetically accessible bryostatin analogues potently induce activation of latent HIV reservoirs in vitro. Nat. Chem. 4(9), 705-710.

52. Pettit, G. R., et al., 1982. Isolation and structure of bryostatin 1. J. Am. Chem. Soc., 104(24), 6846-6848.

53. Lenasi, T., et al., Transcriptional Interference Antagonizes Proviral Gene Expression to Promote HIV Latency. Cell Host Microbe 4, 123-133 (2008).

What is claimed is:

1. A pharmaceutical composition comprising a therapeutically effective amount of a histone deactylase (HDAC) inhibitor that is selective to Class I HDACs and a protein kinase C (PKC) modulator.

2. The composition of claim 1 wherein the weight ratio of the HDAC inhibitor to the PKC modulator is at least about 1:1, at least about 2:1, at least about 3:1, at least about 5:1, at least about 10:1, at least about 20:1, at least about 50:1, or at least about 100:1.

3. The composition of claim 1 wherein the weight ratio of the HDAC inhibitor to the PKC modulator ranges from about 1:1 to about 200:1, from about 1:1 to about 100:1, from about 1:1 to about 50:1, from about 1:1 to about 20:1, from about 1:1 to about 10:1, from about 1:1 to about 5:1, from about 1:1 to about 3:1, from about 2:1 to about 200:1, from about 2:1 to about 100:1, from about 2:1 to about 50:1, from about 2:1 to about 20:1, from about 2:1 to about 10:1, from about 2:1 to about 5:1, from about 2:1 to about 3:1, from about 3:1 to about 200:1, from about 3:1 to about 100:1, from about 3:1 to about 50:1, from about 3:1 to about 20:1, from about 3:1 to about 10:1, from about 3:1 to about 5:1, from about 5:1 to about 200:1, from about 5:1 to about 100:1, from about 5:1 to about 50:1, from about 5:1 to about 20:1, or from about 5:1 to about 10:1.

4. The composition of any of claims 1 to 3 wherein the selectivity of the HDAC inhibitor to Class I HDACs is at least about 20%, at least about 30%, at least about 40%, at least about 50%, at least about 60%, at least about 70%, at least about 80%, at least about 90%, or at least about 95%.

5. The composition of any of claims 1 to 3 wherein the selectivity of the HDAC inhibitor to Class I HDACs is in the range of from about 20% to about 95%, from about 30% to about 95%, from about 40% to about 95%, from about 50% to about 95%, from about 60% to about 95%, from about 70% to about 95%, from about 80% to about 95%, from about 20% to about 75%, from about 30% to about 75%, from about 40% to about 75%, from about 50% to about 75%, or from about 60% to about 75%.

6. The composition of any of claims 1 to 5 wherein the HDAC inhibitor is a compound of Formula I or pharmaceutically acceptable salt, solvate, prodrug, or stereoisomer thereof 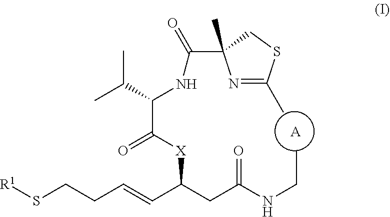
wherein X is O or NH;
R¹ is hydrogen or —C(O)R²;
R² is C₁-C₁₀ alkyl; and
A is 5- or 6-membered heterocyclic ring.

7. The composition of claim 6 wherein R² is methyl, ethyl, propyl, butyl, pentyl, hexyl, or heptyl.

8. The composition of claim 6 or 7 wherein:
A is a 5- or 6-membered nitrogen-containing heterocyclic ring;
A is a 5- or 6-membered sulfur-containing heterocyclic ring;
A is pyridine; or
A is thiazole.

9. The composition of claim 6 wherein the HDAC inhibitor of Formula I is selected from the group consisting of: 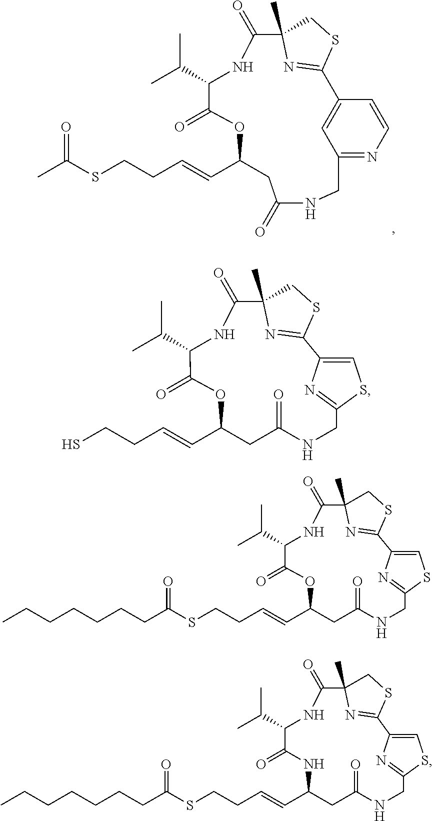
pharmaceutically acceptable salts, solvates, prodrugs, stereoisomers, and mixtures thereof.

10. The composition of any of claims 1 to 9 wherein the PKC modulator comprises bryostatin-1 or an analog thereof.

11. The composition of any of claims 1 to 10 wherein the PKC modulator comprises an analog of bryostatin-1 of Formula IIa or IIb or a pharmaceutically acceptable salt, solvate, prodrug, or stereoisomer thereof: 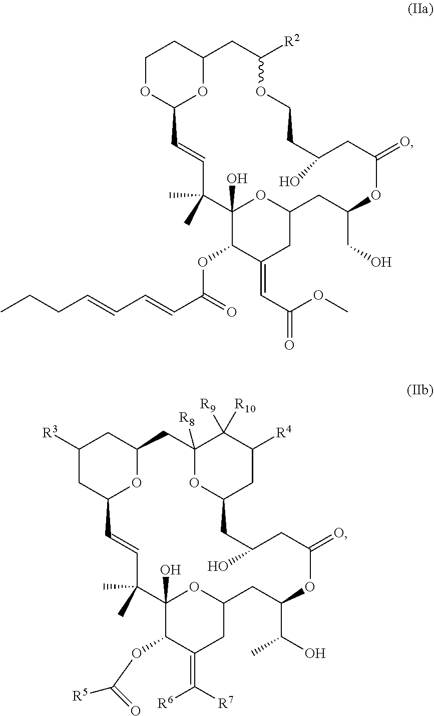
wherein
R² is hydrogen, methyl, ethyl, propyl, isopropyl, tert-butyl, phenyl, or —(CH₂)₃p-Br-Ph
R³ is hydrogen, ═CH₂, ═CHC(O)OCH₂, —CHCO₂CH₃, —CH₂CHCH₂, or —CH₂CO₂(CH₂)₂;
R⁴ is hydrogen, hydroxyl, —OC(O)CH₃, —OC(O)C(CH₃)₃, or —OC(O)C(CH₂)₂CH₃;
R⁵ is hydrogen, —CH₃, —(CH₂)₂CH₃, —(CH)₄(CH₂)₂CH₃, or —C₇C₁₅;
R⁶ and R⁷ are each independently hydrogen or —C(O)OCH₃;
R⁸ is hydrogen or hydroxyl; and
R⁹ and R¹⁰ are each independently hydrogen or methyl.

12. The composition of claim 10 wherein the PKC modulator comprises an analog of bryostatin-1 having the structure:

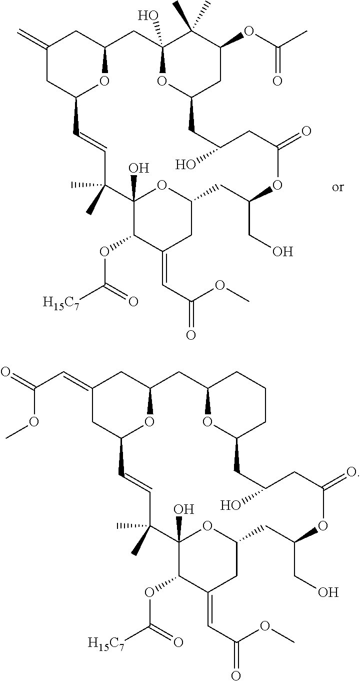

13. The composition of any of claims 1 to 12 wherein the PKC modulator comprises prostratin or an analog thereof.

14. The composition of any of claims 1 to 13 wherein the PKC modulator comprises an analog of prostratin of Formula III or a pharmaceutically acceptable salt, solvate, prodrug, or stereoisomer thereof: 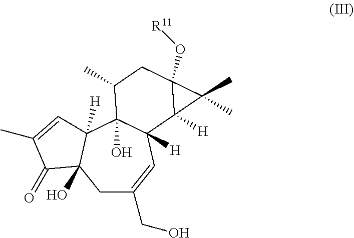
wherein
R¹¹ is C₁-C₆ alkyl (e.g., methyl, ethyl, propyl), —COCH₃, —COCH₂(Ph), or —(CH₂)₂(Ph).

15. The composition of claim 14 wherein R¹¹ is C₁-C₆ alkyl.

16. The composition of any of claims 1 to 15 wherein the composition further comprises at least one pharmaceutically acceptable excipient.

17. A method of treating HIV-1 latency in a subject in need thereof, comprising administering to the subject a pharmaceutical composition of any of claims 1 to 16

18. A method of treating HIV-1 latency in a subject in need thereof, comprising administering to the subject a first pharmaceutical composition comprising a therapeutically effective amount of a histone deactylase (HDAC) inhibitor that is selective to Class I HDACs and a second pharmaceutical composition comprising a therapeutically effective amount of a protein kinase C (PKC) modulator.

19. The method of claim 17 wherein the method further comprises administering an antiviral agent.

20. The method of claim 17 or 18 wherein the subject is a human.

21. A kit comprising a first pharmaceutical composition comprising a therapeutically effective amount of a histone deactylase (HDAC) inhibitor that is selective to Class I HDACs and a second pharmaceutical composition comprising a therapeutically effective amount of a protein kinase C (PKC) modulator.
